(19) United States
(12) Patent Application Publication
(10) Pub. No.: US 2007/0004957 A1
(43) Pub. Date: Jan. 4, 2007

(54) Assembly and method for carrying out magnetotherapy

(76) Inventor: Andreas Hilburg, Oberhausen (DE)

Correspondence Address: MUIRHEAD AND SATURNELLI, LLC, 200 FRIBERG PARKWAY, SUITE 1001, WESTBOROUGH, MA, 01581, US

(21) Appl. No.: 10/543,535

(22) PCT Filed: Jan. 23, 2004
(86) PCT No.: PCT/EP04/50043

§ 371 Date: Aug. 23, 2006

(30) Foreign Application Priority Data
Jan. 31, 2003 (DE) 103 04 085.4

(51) Int. Cl. A61N 2/00

(52) U.S. Cl. 600/9

(57) ABSTRACT

An assembly and a method for carrying out magnetotherapy. Said assembly comprises an application system for applying a magnetic field to a living thing and a control unit for adjusting at least one parameter of the magnetic field. A pulse sensor records a vegetative or motoric function of the living thing and a regulating system adjusts the aforementioned parameter in accordance with the measuring signals from the pulse sensor. According to various embodiments, an aim of the invention is to create a magnetic field, in which the condition of the treated patient is reliably recorded and taken into consideration. To achieve this, the variability of the heart rate is determined from the measuring signals of the pulse sensor.

The invention relates to an assembly for carrying out magnetic field therapy (magnetotherapy) according to the preamble of claim 1 and a method for carrying out magnetic field therapy according to the preamble to claim 13.

Magnetic field therapies, with which an organism, in particular a human organism is exposed to a time-variant magnetic field to increase well-being and stress-relief, are enjoying increasing popularity. The patient is exposed to the magnetic field via an applicator. The applicator has electrical conductors through which a current flows in order to generate the magnetic field. The applicator's conductors are usually integrated in a mat on which the patient to be treated lies.

It has been found that certain low-frequency pulsed electrical currents generate a pulsed magnetic field acting on the patient via the applicator, which, depending upon the parameters of the current flow and hence of the pattern of the magnetic field strength exert different impacts on the patient's organism. A specific pulse shape which is intended to achieve a selective impact in any region of the body is described, for example, in European Patent EP 0 594 655 B1.

Conventional magnetic field therapy devices generate a pulse pattern set by an operator which is issued independently of the actual effect of the magnetic field therapy and the patient's personal state of health.

European Patent EP 0 729 318 describes a device for determining the effect of pulsed magnetic fields on an organism in which an antenna coil or measurement coil is arranged around the coil for generating a primary magnetic field to pick up the secondary field signals which are induced in the measurement coil following each pulse in the primary energy field by means of the secondary and decaying magnetic field arising within the organism. This device may be used to determine the effect of the therapeutic device, namely the intensity of the magnetic field generated in the organism. However, the result of this effect, that is the influence on the human organism resulting from the exposure to the magnetic field, cannot be determined.

It has also been suggested that a biosensor could be attached to the control unit to record a vegetative or motoric function of the living being. The cited publication EP 594.655 B 1 generally describes for example the use of a biofeedback control system for adjusting optimal field parameters of the magnetic field. In one embodiment, a pulse measuring device is used to determine the controlled variables. It states in the description that this is based on the recognition that, if pulse electromagnetic fields are set to have the optimal effect, the pulse rate slows.

However, the heart rate has been found to be less informative with regard to the effect of the magnetic field therapy. Its absolute value and the degree of its change are primarily determined by the physical features and the physical capacity of the living being treated and only to a small degree by the effect of the magnetic field therapy.

The object of the present invention is to create a magnetic field therapy assembly and a magnetic field therapy method in which the condition of the treated patient during the therapy is reliably recorded and taken into consideration.

This object is achieved according to the invention with regard to the assembly by all the features in claim 1 and with regard to the method by all the features of claim 13.

The measuring signals recorded by the pulse sensor are fed to a regulating assembly which set one or more parameters of the magnetic field in accordance with the measuring signals. In a practical embodiment, the regulating assembly is arranged in the control unit. The pulse sensor records the patient's pulse. The heart beat and hence the pulse is one of the essential bioparameters of a human or animal organism. As explained below, valuable findings regarding the state of health of the patient may be derived from the pulse measuring signal.

According to the invention, the heart rate variability is determined from the periodic signal curve of the pulse sensor. The heart rate variability is a measure of the change in the cardiac cycle. The cardiac cycle is the distance between two successive heart beats. In healthy humans, the heart frequency, which when resting is between 60 and 100 beats per minute, normally fluctuates by 15% and more depending upon the respiration. The heart rate changes are the result of a large number of interlinked control circuits in the body which compensate physiological fluctuations. The heart rate change is also called heart rate variability and is a measure for the general adaptability of an organism to internal and external stimuli. It is extremely suitable for evaluating the current physiological condition of the treated living being and of the influence of the therapy on this condition. A more detailed description of the determination and evaluation of the heart rate variability is given below.

If required, other biosensors such as for example measuring electrodes, temperature sensors, resistance sensors, respirometers or respiratory gas analysis systems can be used. These sensors can be used, for example, to determine the following bioparameters: blood pressure, oxygen saturation of the blood, action potentials in the heart (electrocardiogram), potential fluctuations in the brain (electroencephalogram), skin temperature, skin resistance, respiratory rate, respiratory volume and respiratory gas composition.

Since different parameters of the magnetic field and hence of the current fed to the application means influence the patient's state of health, a pulse measurement and analysis to determine the heart rate variability permit a results-based control of the magnetic field therapy. In addition, when setting the parameters of the magnetic field, consideration is taken not only of the direct effects of the magnetic field applicator, namely the strength of the magnetic field applied, but also of other influences on the condition and state of health of the patient, which could be independent of the magnetic field therapy, but which may be read from the pulse sensor's measuring signal.

Like the prior art, the applicator in the assembly according to the invention normally comprises an electrical conductor, which is supplied with current. In a practical embodiment, the control unit influences one or more of the current signal's parameters with which the magnetic field is generated through the applicator. The parameters influenced are, for example, the duration of a single current pulse, the repetition frequency of the individual current pulses within a group of periodic current pulses, the pause, that is the time interval between two successive groups of current pulses, whose reciprocal value is also called the “burst frequency” and the current intensity and current voltage fed to the conductor. The signal pattern and the resulting magnetic field intensity pattern which achieve the desired effect on the patient are dealt with in detail in the literature on magnetic field therapy. One example of this is the above-cited EP 0 594 655 B 1. The information obtained from the pulse sensor's measuring signal may be used to vary the signal parameters to achieve the desired and, on the basis of the measuring signal, sensible therapeutic result.

In a practical embodiment, the sensor for recording the cardiac rhythm, that is the patient's heart beat or pulse, is a known pulse oximeter sensor. Pulsoximetry is a method for determining the oxygen content (02 content) of the blood. Here, a photometric measuring method is used. The color of the blood changes in dependence on whether oxygen is bound in the haemoglobin in the blood (oxyhaemoglobin). Blood with a high oxygen content is reddish in color, while, on the other hand, the color of deoxygenated blood changes to a bluish hue. An oximeter measures the change in the color of the blood. Here, a light source in the oximeter irradiates a section of the patient's body containing blood vessels with light. The heart beat and the blood pressure which varies with the heart beat cause a change in the dilation of the vessels. This rhythmic dilation and contraction of the vessels result in a signal with the rhythm of the heart beat. The pulsating, i.e. variable, part of the recorded signal may be attributed to the blood flowing in the arteries so that the static part of the measuring signal can be subtracted and the variable part used to determine the color and the O2 saturation. For the purposes of this application, it is not mainly the O2 saturation but the dynamic pattern of the detector signal that is processed. Obviously, the O2 saturation can also be used to influence the parameters of the magnetic field. However, in this case, the emphasis is on controlling the parameters by means of the pulse signal recorded.

The heart rate variability is substantially influenced by the two cardiac nerves—sympathetic nerve and parasympathetic nerve (nervus vagus). These influence the heart function, whereby the heart rate is reduced by the parasympathetic nerve and increased by the sympathetic nerve.

During an analysis of the heart rate variability, the cardiac cycle is determined over a specific time interval (for example, 1 or 2 minutes) of the pulse signals. The cardiac cycle is the reciprocal value of the pulse rate and establishes the time interval between two pulse beats, i.e. between two adjacent maxima of the pulse sensor signal. In English, the cardiac cycle is known as the “interbeat interval (IBI)”. A continuous sequence of the cardiac cycles plotted next to each other is called a tachogram. To determine the heart rate variability, the frequency components contained in this tachogram are subjected to a spectral analysis, that is, the amplitude of every frequency component is determined. The low frequency components of the frequency spectrum (LF=low frequency=0.04-0.15 Hz) are primarily attributed to the influences of the sympathetic nerve. The high frequency components (HF=high frequency=0.15-0.5 Hz) are primarily attributed to the influences of the parasympathetic nerve. The quotient of the LF and HF components is considered to be an indicator of sympaticovagal activity. The LF component comprises the integral of the amplitudes in the low-frequency range from 0.04 to 0.15 Hz. The HF component comprises the integral of the amplitudes in the high-frequency range between 0.15 and 0.4 Hz. In normal conditions, the value of said quotients is normally between 1.5 and 2. Usually, the aim is to bring the patient into this normal condition. If the influence of the sympathetic nerve predominates, the patient should be subjected to a sedative influence in order to achieve a normal condition. If the influence of the parasympathetic nerve predominates, a tonicizing program should be selected to bring the patient into a normal condition.

The parameters of the magnetic field therapy are set in accordance with the heart rate variability quotients determined. A first set of parameters is assigned, for example, to a tonicizing magnetic field therapy and a second set of parameters to a sedating magnetic field therapy. In dependence on the heart rate variability quotients, the individual parameters are interpolated between their respective value from the first set of parameters and their respective value from the second set of parameters. For purposes of the interpolation, the heart rate variability may be scaled and standardized so that it lies within a range between 0 and 1, whereby the value 0 is assigned, for example, to the sedative program and the value 1 to the tonicizing program.

The selected example of the parameter control between two concrete parameter sets may obviously be expanded. For example, it is possible to use several parameter sets with tonicizing or sedative influences of different degrees and optionally other therapeutic effects, whereby as a result of the variable derived from the pulse sequence, it is possible to choose, and if necessary interpolate, between these several parameter sets. In addition to the above-described heart rate variability quotients, it also is possible to determine another or an additional indicator from the pulse sensor's measuring signal with which the parameters for generating the magnetic field are influenced. When a pulse oximeter sensor is used, as mentioned above, the oxygen content of the blood can be analyzed and used as an indicator for setting the magnetic field parameters.

In a practical embodiment for executing the method according to the invention, the assembly according to the invention has a circular buffer memory in which, starting from the most up-to-date measuring signals, the temporal pulse sequence over a specific preceding time is stored. Therefore, starting from the current time, the circular buffer memory stores a segment of the preceding time, for example 1 minute of the pulse sequence. As described above, the heart rate variability quotient representative of the cardiac cycle fluctuations is determined from this stored signal.

As mentioned above, pulse signal evaluation is only one of several possibilities for controlling the magnetic field therapy by means of a bioparameter. Alternatively or additionally, it is also possible to use other variables and features of the signal recorded, for example, the type of respiration, oxygen saturation of the blood and other generally known variables derived from the aforementioned bioparameters.

The regulating assembly according to the invention can obviously control not only a magnetic field therapy device but also an additional therapeutic device, which is connected to the same control unit. Suitable as additional therapeutic devices are, for example, sound generating means for audio and sound therapy, light generating means for color and light therapy, electrodes for electrostimulation therapy, devices for generating electrical alternating fields (frequency therapy devices), vibration therapy devices which generate mechanical vibrations, thermal radiators for thermotherapy and oxygen therapy devices.

The following describes an embodiment of the invention in conjunction with the attached drawings in which:

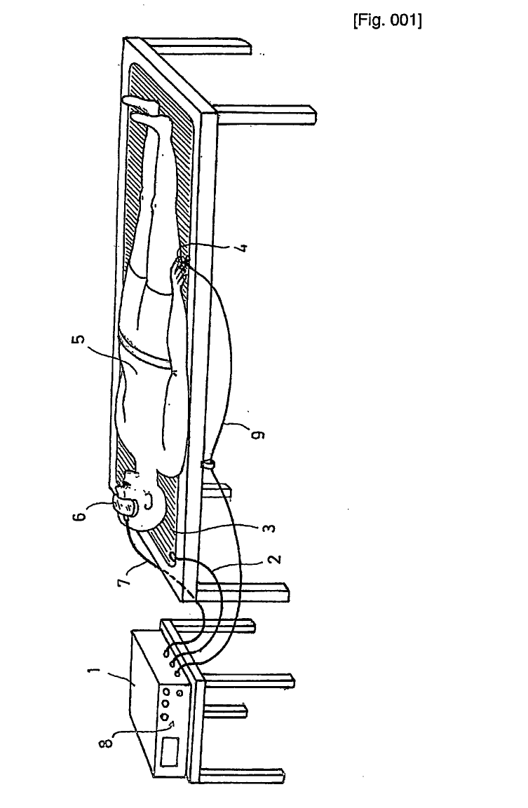

FIG. 1 is a diagrammatical representation of the therapeutic assembly

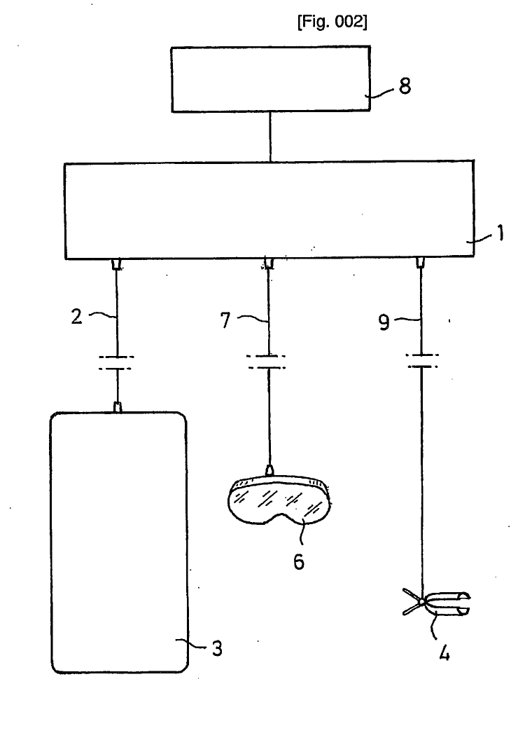

FIG. 2 is a diagram showing the individual components of the assembly

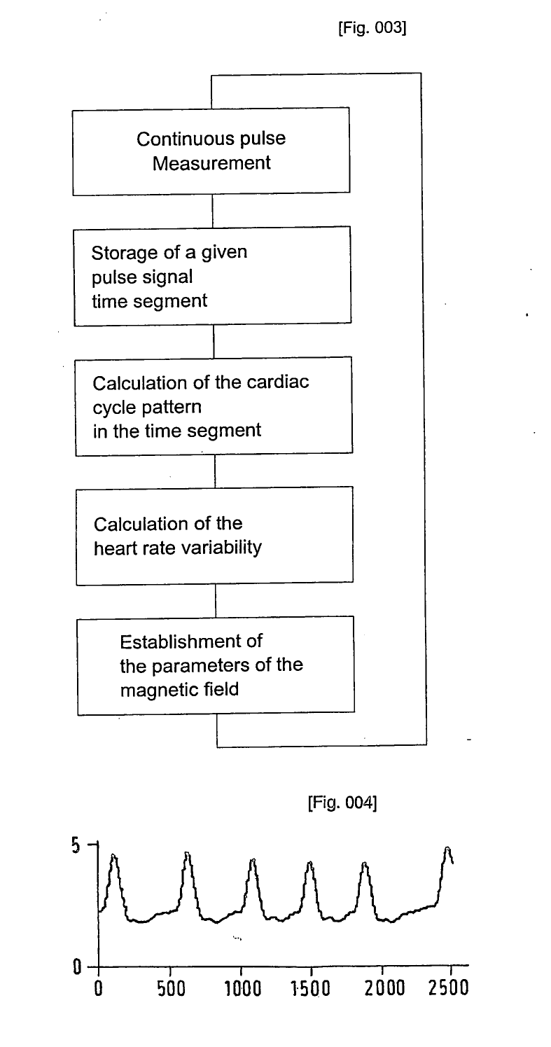

FIG. 3 is flow diagram of the method according to the invention

FIG. 4 is a schematic diagram of a measured pulse curve

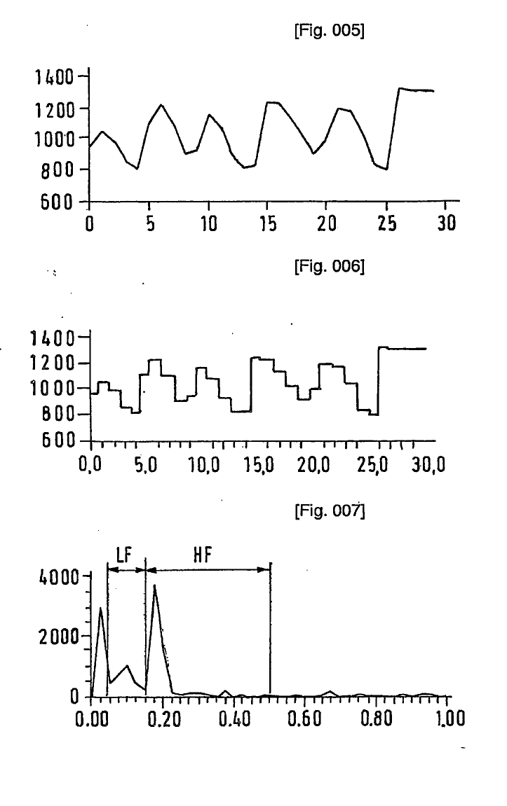

FIG. 5 is a cardiac cycle series derived from the pulse curve

FIG. 6 is a corrected cardiac cycle series in which artefacts, that is artificial influences on the measuring signal, have been filtered out and

FIG. 7 is the result of a spectral analysis of the cardiac cycle signal.

FIG. 1 shows the magnetic field therapy assembly with a control unit 1 to which a magnetic field mat 3 is connected by means of a connection cable 2. The magnetic field mat 3 contains a number of electrical conductors with an electrically conductive connection to the control unit 1 by means of the connection cable 2. The control unit 1 directs a current into the electrical conductors in the magnetic field mat 3 which generates a magnetic field over the magnetic field mat 3. In a practical embodiment, the current is time-variable and proceeds in individual pulses which are combined in pulse groups which are each separated by pauses between two pulse groups. The shape and frequency of the individual current pulses, the time-variable amplitude-pattern of the current pulses and the pauses between the successive pulse groups (the reciprocal value of the pulse group period is called the burst frequency) have a significant influence on the effect of the magnetic field on the patient's organism. Normally a fixed value for these parameters is entered on the control unit or a predefined sequence of these parameters is selected in order to achieve a specific therapeutic effect.

In the assembly according to the invention, a finger sensor 4 is provided which is used to record measuring signals representing the pulse of the patient 5 and feed them to the control unit 1. The control unit 1 can use these pulse signals to determine one or more indicators with which the parameters of the magnetic field are regulated.

FIG. 1 shows another therapeutic device 6 in the form of color therapy goggles, which screen the eyes of the patient 5 from exposure to external light and in which heterochromatic light is generated in pulses and with color changes in order to assist the therapeutic effect of the magnetic field mat 3. The color therapy goggles 6 are also connected to the control unit by means of a connection cable 7. In the case of an autonomous power supply, the therapeutic devices (magnetic field mat 3 and color therapy goggles 6) can also be addressed by the control unit 1 via a cable-less data connection (for example Bluetooth or wireless LAN).

FIG. 2 shows the components of the therapeutic assembly according to the invention. The core is the control unit 1 which has a control console 8 on either its front side or its top side or is connected by a data link with a control console 8 of this kind. The control console 8 is equipped with switches and buttons for adjusting the control unit 1. It also has analog or digital display devices showing the settings of the control device 1.

The magnetic field mat 3 which forms the therapeutic arrangement's application device is connected to the control unit 1 by means of a connection cable 2. The control unit 1 generates a specific current flow whose parameters can be set in accordance with the desired therapeutic effect. The current is guided through the conductors in the magnetic field mat 3 in such a way that a magnetic field forms around these conductors with parameters directly determined by the parameters of the introduced current.

Another therapeutic device 6, for example the color therapy goggles shown in FIG. 1 is connected to the control unit by the second connection cable 7 and also receives control currents to generate the therapeutic effect of the therapeutic device 6.

To record the pulse of the patient 5, a pulse oximeter sensor 4 namely a finger sensor, which functions in the way described above, is connected to the control unit 1. The signal cable 9, which connects the pulse oximeter sensor 4 with the control unit 1 on the one hand supplies the supply voltage for the light source in the pulse oximeter sensor 4 and on the other hand forwards the measuring signals from the detector in the pulse oximeter sensor 4 to the control unit 1. The control unit 1 uses the indicators derived from the pulse signal to control the parameters of the current fed to the magnetic field mat 3.

FIG. 3 is a flow diagram of this control process. The measuring signals from the continuous pulse measurement of the pulse oximeter sensor 4 are stored. A circular buffer memory is provided for this which in each case stores a prespecified time segment starting from the most recent measuring signals. During this, the respective oldest stored signals are overwritten by the respective most recent signals so that the same time segment, starting from the most recent measuring signal, is stored at each point in time. A graphical representation of the pulse signal recorded is shown in FIG. 4.

The course of the cardiac cycle is calculated from the pulse sensor's measuring signal. The cardiac cycle (inter beat interval) is defined as the distance between two successive maxima of the pulse curve and represents the time between two heart beats. The sequence of the successive cardiac cycles as calculated from a pulse curve is shown in FIG. 5 as a tachogram. This tachogram is subjected to an artefact correction to produce a tachogram as an equidistant series in time in which the preceding cardiac cycle is depicted for every heart beat (see FIG. 6).

The tachogram in FIG. 6 is subjected to a spectral analysis or frequency analysis whereby the respective amplitudes for the different frequency components of the tachogram are displayed. Analytical procedures of this kind are known from in the art. One example, is the Fast Fourier transformation.

The result of the spectral analysis is shown in FIG. 7. As described above, it is possible to define a heart rate variability quotient which is obtained from the quotient between the low-frequency component and high-frequency component of the spectral analysis. The low-frequency component (LF=low frequency) is calculated as the integral of the amplitudes in the range between 0.04 and 0.15 Hz. The high-frequency component (HF=high frequency) is calculated as the integral of the amplitudes in the interval between 0.15 and 0.4 Hz. This quotient is used to control the therapeutic device. Precise interpretations of the findings and information on the measuring procedures may be found in the specialist literature, for example in the publications of the task force of the European Society of Cardiology and the North American Society of Pacing and Electrophysiology: Heart rate variability. Standards of measurement, physiological interpretation, and clinical use. Circulation 1996 (93) 1043-1065.

The recorded measuring signals can obviously also be used to control the other therapeutic devices, for example the color therapy goggles 6. It is also possible to derive other indicators from the measured signals in addition to the mentioned heart rate variability.

Even though the invention is primarily described with reference to the example of pulse measurements and therapy control by means of variables determined from the heart rate variability, it is not restricted to this. As mentioned at the start, the therapy may be controlled with a plurality of different sensors which record different bioparameters taking into consideration various variables derived therefrom.

### LIST OF REFERENCE NUMBERS

1 Control unit
2 Connection cable
3 Application means, magnetic field mat
4 Pulse oximeter sensor, finger sensor
5 Patient
6 Color therapy goggles, color therapy device
7 Connection cable
8 Control console
9 Signal cable

1. Assembly for carrying out magnetic field therapy, comprising:
an application means for applying a magnetic field to a living being;
a control unit for adjusting at least one parameter of the magnetic field;
a pulse sensor for recording the pulse of the living being;
a regulating assembly for adjusting said parameter in accordance with measuring signals of the pulse sensor; and
a circuit for determining the heart rate variability from the measuring signals of the pulse sensor.

2. Assembly according to claim 1, further comprising:
an additional biosensor for recording at least one of the following vegetative or motoric functions of the living being: blood pressure, oxygen saturation of the blood, action potentials in the heart (electrocardiogram), potential fluctuations in the brain (electroencephalogram), skin temperature, skin resistance, respiratory rate, respiratory volume or respiratory gas composition.

3. Assembly according to claim 2, wherein the additional biosensor is at least one of the following biosensors: measuring electrodes, temperature sensors, resistance sensors, respiratory measuring device or respiratory gas analysis device.

4. Assembly according to claim 1, wherein the control unit and the regulating assembly have electrical circuits for adjusting several parameters of the magnetic field.

5. Assembly according to claim 1, wherein said parameter is a parameter of a current signal which is applied to an electrical conductor in the application means.

6. Assembly according to claim 5, wherein said parameter or parameters are selected from the following group: duration of a current pulse, frequency within a group of current pulses, time interval between two successive current pulse groups, current intensity or voltage.

7. Assembly according to claim 1, wherein the pulse sensor is a pulse oximeter sensor.

8. Assembly according to claim 1, wherein the circuit has a memory to record a segment of a temporal pulse path.

9. Assembly according to claim 8, wherein the memory is a circular buffer memory in which the temporal pulse pattern of a specific preceding period starting from the most up-to-date measuring signals is stored.

10. Assembly according to claim 9, wherein the circuit has a component for determining the temporal course of the cardiac cycle and a component for determining the cardiac cycle fluctuations from the temporal pulse pattern.

11. Assembly according to claim 1, further comprising:
at least one additional therapeutic device which is connected to the control unit.

12. Assembly according to claim 11, wherein the additional therapeutic device is selected from the following group of therapeutic devices: electrostimulation devices, audio and sound therapy devices, light therapy devices, color therapy devices, frequency therapy devices, vibration therapy devices, thermal therapy devices or oxygen therapy devices.

13. Method for generating a magnetic field in which a control unit provides a time-variant current for generating a magnetic field which is supplied to an application means which applies the magnetic field to a living being, wherein the pulse of the living being is recorded by means of a pulse sensor, characterized in that the heart rate variability is determined from the measuring signals of the pulse sensor and the current flow through the control unit is set in accordance with the determined heart rate variability.

14. Method according to claim 13, wherein in addition at least one of the following vegetative or motoric functions of the living being is recorded and used to set the current flow: blood pressure, oxygen saturation of the blood, action potentials in the heart (electrocardiogram), potential fluctuations in the brain (electroencephalogram), skin temperature, skin resistance, respiratory rate, respiratory volume or respiratory gas composition.

15. Method according to claim 13, wherein the control unit sets several parameters of the current flow.

16. Method according to claim 15, wherein the control unit sets at least one parameter from the following group: duration of a current pulse, frequency within a group of current pulses, time interval between two successive current pulse groups or current intensity or voltage.

17. Method according to claim 13, wherein a pulse oximeter sensor is used as a biosensor.

18. Method according to claim 17, wherein a finger sensor or ear clip sensor is used.

19. Method according to claim 13, further comprising:
one segment of the temporal pulse pattern is stored in a memory;
the temporal pattern of the cardiac cycle is determined from the stored segment of the temporal pulse pattern; and
the cardiac cycle fluctuations are determined from the stored segment of the temporal pulse pattern.

20. Method according to claim 19, wherein a specific preceding time period is stored in a circular buffer memory starting from the most up-to-date measuring signals.

21. Method according to claim 19, wherein the cardiac cycle fluctuations are determined by a frequency analysis of the temporal course of the cardiac cycle.

22. Method according to claim 13, wherein the control unit uses the measuring signals from the biosensors to establish a set with several parameters, and wherein at least two support points are defined for each parameter and interpolation between the support points is performed in accordance with the measuring signals of the pulse sensor.

23. Method according to claim 13, wherein at least one additional therapeutic device controlled by the control unit acts on the living being.

24. Method according to claim 23, wherein the additional therapeutic device is selected from the following group: electrostimulation devices, audio and sound therapy devices, light therapy devices, color therapy devices, frequency therapy devices, vibration therapy devices, thermal therapy devices or oxygen therapy devices.

25. Assembly according to claim 7, wherein said pulse oximeter sensor is a finger sensor or an ear clip sensor.

26. A magnetic field device, comprising:
an applicator that applies a magnetic field;
a control unit that adjusts at least one parameter of the magnetic field;
a sensor that records measured information;
a regulating assembly that adjusts said at least one parameter in accordance with measuring signals of the sensor; and
a circuit that determines variability of the measuring signals of the sensor.
